(19) United States
(12) Patent Application Publication
(10) Pub. No.: US 2004/0043476 A1
(43) Pub. Date: Mar. 4, 2004

(54) Multi-stage malodorous or noxious gas purification system

(76) Inventors: Suk Ku Sim, Daejeon (KR); Eun Kwan Lee, Kyunggi-do (KR)

Correspondence Address: NIXON & VANDERHYE, PC, 1100 N GLEBE ROAD, 8TH FLOOR, ARLINGTON, VA, 22201-4714, US

(21) Appl. No.: 10/432,369

(22) PCT Filed: Nov. 23, 2001
(86) PCT No.: PCT/KR01/02025

(30) Foreign Application Priority Data
Nov. 24, 2000 (KR) 2000/70166

(51) Int. Cl. A61L 9/01

(52) U.S. Cl. 435/266

(57) ABSTRACT

A multi-stage malodorous or noxious gas purification system comprises: purifiers for extracting unnecessary ingredients from gas including such unnecessary ingredients; a plurality of unitary purification tanks each having a filling space for filling the purifiers therein and an inlet opening and an exit opening for the gas to pass through the filling space; and at least one connecting member being gas-permeable and installed at the inlet opening or the exit opening of each unitary purification tank, for isolatedly connecting the plurality of unitary purification tanks, wherein two or more different kinds of the purifiers are used.

### TECHNICAL FIELD

The present invention generally relates to an apparatus for purifying air, and more specifically to a system for purifying malodorous or noxious gas employing a multi-stage method.

### BACKGROUND ART

In general, malodors produced from domestic electrical appliances, mobile toilets, or in the processes of industrial facilities such as environmental waste treatment facilities are caused by mixed gas mainly comprising hydrogen sulfide H₂S, ammonia NH₃, methylmercaptane CH₃SH, trimethylamine (CH₃)₃N, etc. The causative agents of such malodors and other noxious gas cause pains to people by stimulating the human nerve system, and can give considerable damage to the health of people, which in turn become main reasons for public environmental complaints.

In order to solve such problems of malodorous or noxious gas, various purification techniques are used conventionally, for. example, use of sprays and perfumes to disguise malodors with fragrant smells, absorption of malodorous or noxious gas by using adsorbents such as activated carbon or charcoal, physio-chemical decomposition of the causative agents of malodorous or noxious gas, and use of microorganisms which can decompose the causative agents of malodorous or noxious gas by contacting therewith.

Among such techniques, the use of sprays and perfumes to disguise malodors with fragrant smells is limited to only a small space with temporary effect. The absorption of malodorous or noxious gas by using adsorbents such as activated carbon or the like has an advantage of relatively simple structure and easy maintenance of the apparatus. However, in such a technique, cleansing or replacement of the activated carbon or charcoal is required periodically, and in addition, it is impossible to eliminate stimulative malodorous gas such as ammonia which cannot be adsorbed by the adsorbents. While, in the purification by microorganisms, waste gas is purified through the natural decomposition by microorganisms in the soil or the adsorption by soil particles, by passing the waste gas through a soil layer in which the microorganisms live. This process is quite effective for the purification of high-concentration waste gas, however it requires an enormous amount of installation and maintenance cost including large installation area for the facility.

The purification apparatus using microorganisms improved over such prior art purification systems have been developed, for example, those shown in FIGS. 1 to 3. FIG. 1 shows an embodiment of a Korea Patent (Patent Registration No. KR-0138050, Feb. 16, 1998), entitled “Method and Device for Biologically Deodorizing Waste Gas”, granted to ENVI SYSTEM CO., LTD (Republic of Korea). A microorganism deodorization device 10 shown in FIG. 1 has a deodorization tower 4 including a plurality of serially arranged unitary deodorization tanks 6 containing wood pieces or bark pieces 8 therein. Waste gas is introduced into an inlet opening 4a, passed through a humidistat 2, and purified by the microorganisms in the deodorization tower 4. Then, the gas is passed through a collector 9 and discharged at an exit opening 4b as purified gas. The unitary deodorization tank 6 includes two spaces partitioned by a metal net 6a, and only one of the spaces is filled with wood pieces or bark pieces 8 so that microorganisms can live therein. Each unitary deodorization tank 6 is adjacent one another with a cross-plate 6b positioned therebetween, the cross-plate 6b having vents formed therein.

As described above, the deodorization device 10 is configured such that the microorganisms are disposed in multi-layers so as to increase general gas permeability, thereby increasing deodorization effect. In the device 10, nonetheless, it is difficult to remove and replenish the microorganisms and wood pieces which are the nutritional source for the microorganisms since there is no apparatus for separately feeding or supplying such microorganisms and wood pieces. Further, separation effect of microorganisms can be hardly achieved because there is a high possibility that the microorganisms can move from one layer to another through the openings or vents in the metal net 6a or the cross-plate 6b and be mixed together. In addition, there is no description about the separation of microorganisms in accordance with their species in the Patent document..

That is, it is necessary to mix various species of microorganisms to purify composite waste gas including causative agents of various malodorous or noxious gases since usually one species of microorganisms has capability to decompose one type of compounds. In the meanwhile, if different species of microorganisms are mixed one another without separation, some struggle between different species can occur, which in turn significantly lowers the efficiency of purification. Moreover, the deodorization device 10 is not flexible to employ various effective microorganisms for purification of waste gas—for example, the species of microorganisms that do not feed on wood pieces, but are greatly effective in deodorization—since the nutritional source for the microorganisms is limited only to wooden materials in the description of the document.

Additionally, there are other examples that include a prior art apparatus for supplying a malodorous or noxious gas purification system with microorganisms and culturing solution shown in FIGS. 2 and 3. FIG. 2 shows a deodorization apparatus 20 of a Korea Patent Application No. 1999-0009239 (filed on Mar. 18, 1999), entitled “Apparatus for Removing Unpleasant Odor and Volatile Organic Compounds Using Microorganism”, by ENBIOTEC CO., LTD (Republic of Korea), and FIG. 3 shows a purification device 30 of a Korea Patent Application No. 2000-0023935 (filed on May 4, 2000), entitled “Deodorizing Device Using Microbe”, by WORLD INDUSTRIAL MACHINERY CO., LTD (Republic of Korea).

The deodorization apparatus 20 in FIG. 2 is constructed such that waste gas is introduced into an inlet opening 21a of a decomposition tank 21, diffused by porous substances 24, deodorized by carbon filters 23 in which microorganisms live, and then discharged at an exit opening 21b as purified gas. In the deodorization apparatus 20, the microorganisms in a microorganism storage tank 26 are supplied by a pump 28, and these microorganisms are mixed with neutralizing agents and nutritional agents 29 by passing through a mixer 27 and are injected via nozzles 22 installed within the tank 21. Into the storage tank 26, microorganisms cultured at culturing media 25 are fed. The above-described deodorization apparatus 20 has an advantage of easy maintenance of the malodorous or noxious gas purification system, since the proper pH of the microorganisms is controlled and the microorganisms are fed automatically. However, the deodorization apparatus 20 is still not quite satisfactory to apply to composite malodorous gas, because the decomposition tank 21 in which purifying action takes place is not configured as multi-stages. In addition, there is no specific alternative to cause the microorganisms and nutritional agents to remain in the carbon filters 23 for a prolonged time.

While, in the purification device 30 of the prior art shown in FIG. 3, waste gas is sucked through an inlet tube 32a, arrives at and purified by a deodorization unit 31, and then the purified gas is discharged through an exit tube 32b into the air. Within the deodorization unit 31, filter media 33 divided into two strata are installed. Microorganisms from a microorganism culture section 36 and culturing solution from a storage tank 39 are fed into the filter media 33, through injection nozzles 32. For this device 30, though the deodorization unit 31 is constructed as two stratum filter media 33, there is no description on the separation of different species of microorganisms within respective filter media 33. In addition, although the microorganisms are inhabited in the filter media 33, there is a high possibility that the microorganisms injected along with culturing solution cannot remain in the filter media for a long time and might be washed away since the filter media 33 used in this case are made of the same material as common air filters. Therefore, the living environment is not quite favorable for the existence of microorganisms.

Accordingly, there is a need for a malodorous or noxious gas purification system in which purification capability of microorganisms which purify malodorous or noxious gas can be effected, by providing habitat spaces for each species of microorganisms, separating/ isolating respective habitat spaces for each species of microorganisms, thereby improving air permeability between the habitat spaces, so as to effectively remove composite causative agents of various malodorous or noxious gas.

### DISCLOSURE OF INVENTION

The present invention is designed to solve the problems of the prior art malodorous or noxious gas purification systems.

Therefore, it is an object of the present invention to provide a malodorous or noxious gas purification system which can purify malodorous or noxious gas in multi-stages, by isolating and separating one species of microorganisms effective in decomposition of specific ingredients among causative agents of composite malodorous or noxious gas from another species of microorganisms, and at the same time, providing environment suitable for culturing such microorganisms within the purification system.

In order to accomplish the object, a multi-stage malodorous or noxious gas purification system according to the present invention comprises: purifiers for extracting unnecessary ingredients from gas; two or more unitary purification tanks each having a filling space for filling the purifiers therein, each unitary purification tank having an inlet opening and an exit opening for the gas to pass into or out of the filling space; and at least one connecting member for isolatedly connecting the filling spaces of the two or more unitary tanks and for the gas to pass therethrough, each connecting member installed at the inlet opening or the exit opening of the respective unitary purification tanks.

It is preferable to use two or more different kinds of purifiers. At least one kind of the purifiers is a porous substance to adsorb predetermined ingredients among the unnecessary ingredients, or includes microorganisms which can decompose predetermined ingredients among the unnecessary ingredients.

Furthermore, the purifier including microorganisms can comprise habitat members for providing environment for the microorganisms to live in, specific species of microorganisms living in the habitat members, and organic substances as a nutritional source for the microorganisms. The nutritional source for the microorganisms can be fed in liquid with organic substances dissolved. The habitat members, which also serve as the porous substance to adsorb predetermined ingredients, can be carriers consisting mainly of carbon, and preferably, the habitat members can be made of a micro-porous substance with fine pores formed therein, whose size is in the order of several microns.

In addition, each unitary purification tank has a gas-permeable intermediate filter for dividing each filling space into two partitioned spaces. One of the two partitioned spaces can be constructed to be filled with the purifiers. Alternatively, the unitary purification tank further includes a gas-permeable opening filter at the inlet opening or the exit opening so that the space between the opening filter and the intermediate filter can be filled with the purifiers. Additionally, a plurality of inlet openings and exit openings can be formed at the unitary purification tanks.

A closable aperture can be further formed at each unitary purification tank for injecting the purifier therethrough. Furthermore, the unitary purification tank may further comprise an apparatus for automatically feeding microorganisms and culturing solution including organic substances which are the nutritional source for the microorganisms, the apparatus connected to each unitary purification tank.

Each connecting member may comprise a connecting tube one end of which is communicably connected to the inlet opening of one unitary purification tank and the other end of which is communicably connected to the exit opening of another unitary purification tank, and a switch for opening and closing the connecting tube. In addition, the unitary purification tank may further comprise an air blower for forcibly controlling the flow of air passing through the unitary purification tank.

In the multi-stage malodorous or noxious gas purification system in accordance with the present invention, the unitary purification tanks can be arranged serially in one dimension, or may be arranged in two or three dimensions. For one-dimensional serial arrangement, the unitary purification tank may further comprise a chimney-type housing.

### BRIEF DESCRIPTION OF THE DRAWINGS

These and other features, aspects, and advantages of the present invention will be fully understood with reference to the following description, appended claims, and accompanying drawings, in which like numerals refer to like components. In the drawings:

FIG. 1 is a schematic view of an embodiment of a prior art multi-layer waste gas purification apparatus;

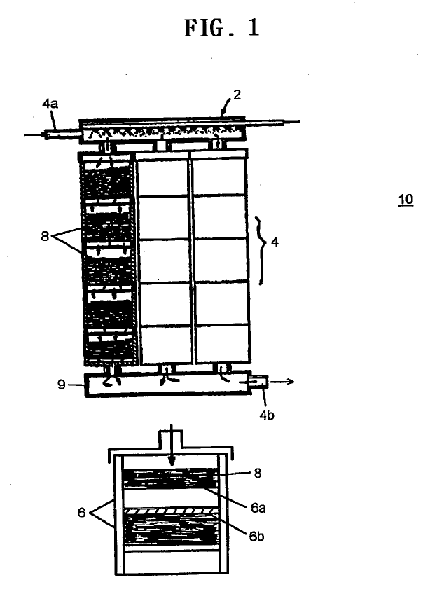

FIG. 2 is a schematic view of an embodiment of a prior art purification apparatus using microorganisms;

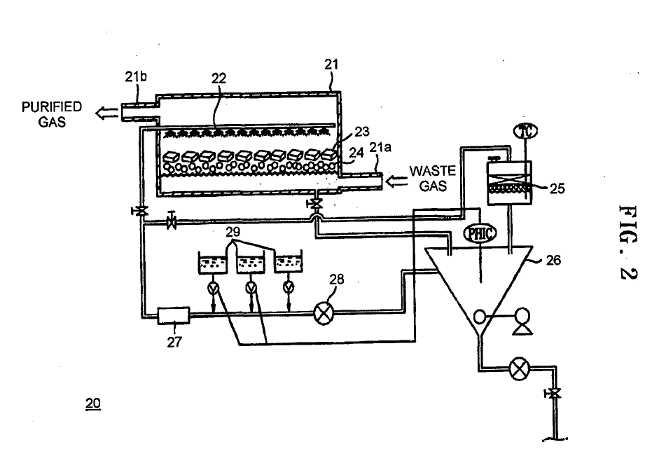

FIG. 3 is a schematic view of an embodiment of another prior art purification apparatus using microorganisms;

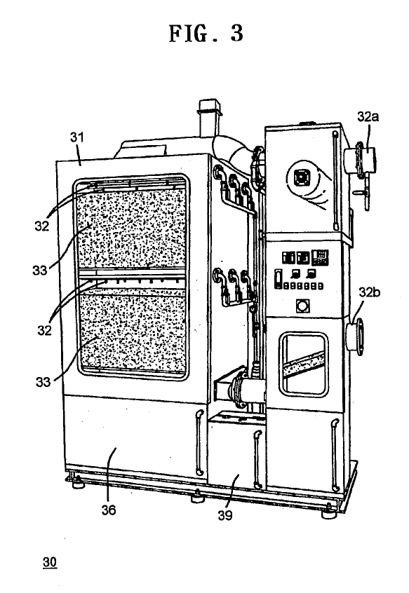

FIG. 4 is a schematic view of a multi-stage malodorous or noxious gas purification system in accordance with a preferred embodiment of the present invention;

FIG. 5 is a schematic view of a unitary purification tank employed in the multi-stage malodorous or noxious gas purification system in accordance with the invention;

FIG. 6 is respectively a section view and an exploded view of an embodiment of a structure of a filter assembly of the unitary purification tank in accordance with the invention;

FIG. 7 is respectively a section view and an exploded View of an embodiment of a structure of another filter assembly of the unitary purification tank in accordance with the invention;

FIG. 8 is a schematic view of a multi-stage malodorous or noxious gas purification system with a purification path control unit according to the invention;

FIG. 9 is a schematic view of a multi-stage malodorous or noxious gas purification system with an automatic feed apparatus for microorganisms and culturing solution in accordance with the invention;

FIG. 10 is a block diagram illustrating the control relationship of a multi-stage malodorous or noxious gas purification system in accordance with a preferred embodiment of the invention;

FIG. 11 is a schematic view of a multi-stage malodorous or noxious gas purification system configured in chimney-type in accordance with an embodiment of the invention; and

FIG. 12 is a schematic view of a multi-stage malodorous or noxious gas purification system configured in two dimensions in accordance with another embodiment of the invention.

### BEST MODE FOR CARRYING OUT THE INVENTION

Hereinafter, preferred embodiments of the present invention will be described in detail with reference to the accompanying drawings.

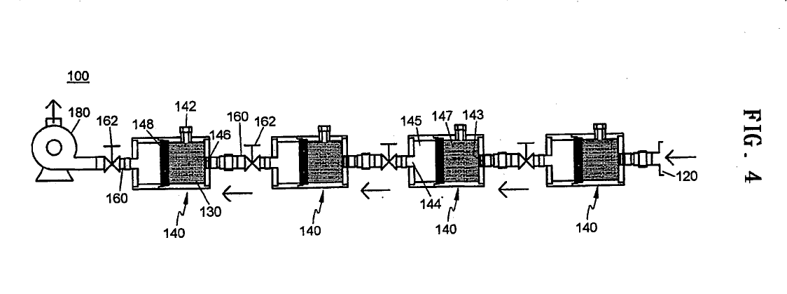

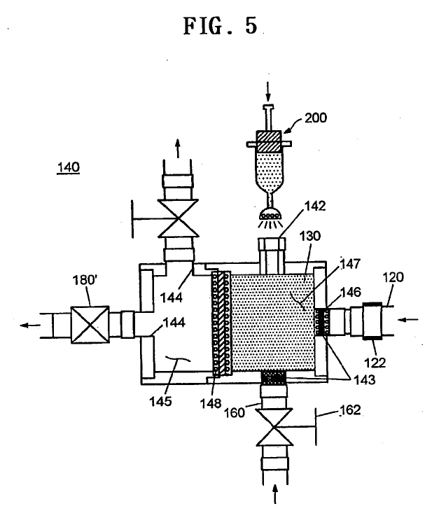

FIG. 4 shows a multi-stage malodorous or noxious gas purification system 100 in accordance with a preferred embodiment of the present invention, and FIG. 5 shows an embodiment of a unitary purification tank. 140 which can be employed in the multi-stage malodorous or noxious gas purification system.

The multi-stage malodorous or noxious gas purification system 100 as shown in FIG. 4 is used for removing unnecessary ingredients from air, such as causative agents of malodorous or noxious gas contained in the air, and can comprise purifiers, a plurality of unitary purification tanks 140, and at least one connecting member 160. The present invention has a structural characteristic in that within each unitary purification tank 140 the purifiers are filled, and in that each of the unitary purification tanks is isolatedly connected with a gas-permeable connecting member 160.

In a further preferred embodiment, two or more different kinds of purifiers can be used. That is, one of the unitary purification tanks 140 can be filled with a substance that can adsorb the causative agents of malodorous or noxious gas, and another unitary purification tank 140 can be filled with cultured microorganisms that have capability to decompose the causative agents of malodorous or noxious gas. Furthermore, the number of such unitary purification tanks can be 4 or 10 without limitation, as required. Therefore, the present invention can provide a malodorous or noxious gas purification system that can effectively remove and purify causative agents of malodorous or noxious gas, or various causative agents causing air pollution.

As purifiers to extract unnecessary ingredients from the gas to be purified, any substances that purify air can be used. The causative agents of malodors or air pollution exist mixedly in the form of dust or chemical gas in the polluted air. Such causative agents comprise hydrogen sulfide H₂S, ammonia NH₃, methylmercaptane CH₃SH, trimethylamine (CH₃)₃N, etc. Accordingly, it is preferable to select purifiers that are most effective in removing each causative agent and to fill separately such purifiers into respective unitary purification tanks.

That is, at least one kind of the purifiers to be filled in respective unitary purification tanks 140 is a porous substance that can adsorb and collect predetermined ingredients among the unnecessary ingredients so as to purify the air. For. example, adsorbents consisting of wood charcoal or activated carbon can be employed, or common air filters can be used. In addition, it is preferable that at least one kind of the purifiers is a purifier including microorganisms which decompose predetermined ingredients of the unnecessary ingredients. In a still preferred embodiment, the unitary purification tanks, respectively, filled with an adsorbent consisting of the porous substance and a purifier including microorganisms can be properly arranged in stages.

The purifier including microorganisms in accordance with a preferred embodiment of the invention can comprise habitat members, microorganisms and organic substances. The habitat members are a means to provide environment for certain species of microorganisms to live in for a predetermined or semi-permanent period of time, and are made of inorganic substances, preferably, those not affected by the decomposition of corresponding microorganisms. The habitat members also serve as the porous substance to adsorb predetermined ingredients. As a matter of course, it is also possible to use any substances that cannot be decomposed by the microorganisms as habitat members, even though the substances include organic substances. Any microorganisms that have capability to decompose and convert certain chemical substances, especially, gases harmful to human bodies or chemical gases causative of malodors, into un-harmful and non-odorous gas can be employed. Any kind of organic substances that can be decomposed and be the nutritional source for the microorganisms can be also used.

In general, hydrogen sulfide H₂S, ammonia NH₃, methyl mercaptane CH₃SH, trimethylamine (CH₃)₃N, etc. are known as causative agents of malodors. Therefore, it is possible to use certain species of microorganisms that can decompose any one chemical substance among such causative agents of malodors. Since the present malodorous or noxious gas purification system is configured in multi-stages, the system has an effect to remove all, for example four, causative agents of malodorous gas, by separately accommodating each species of microorganisms capable of decomposing specific chemical substances among the causative agents in each stage, and passing malodorous gas through the multi-stages, that is, sequentially passing the malodorous gas through the four unitary purification tanks having respectively four different species of microorganisms therein. Accordingly, there is no need to use microorganisms capable of decomposing simultaneously several causative agents, but it is possible to use microorganisms capable of decomposing just one causative agent, thereby improving the purification efficiency of malodorous or noxious gas.

The organic substances provided as the nutritional source for the microorganisms can be fed in the form of solid pieces or powder. However, it is preferable to feed the organic substances in liquid into which the organic substances are dissolved with water or other solvent. For example, it is possible to spray a culturing solution to the spaces in which the microorganisms live with spray devices. In such spray of organic substances in liquid state, it is easy to adjust an amount of feed, furthermore, it is possible to preclude a possibility that the remaining organic substances become a cause of malodors. In addition, in case the culturing solution is fed in liquid state, since it is easy to mix the microorganisms with the culturing solution, the only thing to do for replacement of purifiers is to mix the microorganisms with the culturing solution in a predetermined ratio and to spray the mixed solution into the unitary purification tanks in a predetermined period of time.

For the habitat members, it is possible to use various substances, for example, plastic materials in the form of woven fabric or sponge, or glass or ceramic materials in the form of fine granules. It is preferable that the size of the granules is more than about 4 mm in diameter, such that the granules cannot be slipped away through the filters or other separation members. In the meanwhile, the habitat members are preferably provided with openings for the gas to pass therethrough.

As the habitat members, a porous substance consisting mainly of carbon and serving to adsorb predetermined ingredients can be also employed. When such porous substance of carbon, for example, wood charcoal or activated carbon is used, the efficiency of air purification is significantly increased due to the combination of the adsorption by the porous substance with the decomposition by the microorganisms. In addition, as is well known in the art, such porous substance of carbon can be an optimum habitat space for the microorganisms, since the microorganisms live well in a porous carrier consisting of carbon components.

Further, the openings formed in the porous substance consisting of carbon are preferably of the size of a few microns. Such openings of the size with a few microns are similar with the size of common microorganisms, so as to be an optimum habitat space especially for microorganisms, and there is little chance for the microorganisms to slip away through the fine openings. In this case, it can be far easier to make a structure that can perfectly isolate specific species of microorganisms within each unitary purification tank and allow only gas to pass therethrough.

The unitary purification tanks 140 of the multi-stage malodorous or noxious gas purification system 100 shown in FIG. 4 have an essentially identical structure. On the other hand, the unitary purification system 140 can be configured differently, as shown in FIG. 5. Each unitary purification tank 140 has a basic structure comprising a filling space for filling various above-mentioned purifiers therein, and an inlet opening and an exit opening for the gas to pass through the filling space.

In accordance with a preferred embodiment, the unitary purification tank is configured such that the filling space is divided into at least two spaces by a partition. This is for better gas-permeability. Preferably, one of the divided spaces becomes a space 147 to be filled with the purifiers, and the other of the divided spaces is a buffer space 145 which is kept empty. In this case, the partition may consist of a filter, or an intermediate filter 148 which allows only gas to pass therethrough. The intermediate filter 148 makes the purifiers remain within the space 147, and may consist of gas-permeable material. However, the intermediate filter 148 may have an additional function to adsorb causative agents of a certain kind of malodorous or noxious gas.

Further, the unitary purification tank 140 further comprises a gas-permeable filter or inlet filter 146 at an inlet opening 143 or another filter at an exit opening 144, and the space between the intermediate filter 148 and the inlet filter 146 is preferably filled with the purifiers. Such configuration is desirable especially for the case when microorganisms are used as a purifier, this is because the microorganisms can be filled between the two filters such that the separation effect is increased.

As shown in FIG. 5, a unitary purification tank in accordance with another preferred embodiment of the invention may comprise a plurality of inlet openings 143 and a plurality of exit openings 144. The inlet openings 143 are formed with respect to the space 147 filled with the purifiers, and the exit openings 144 are formed with respect to the buffer space 145 with no purifier therein. The number of the inlet opening 143 and the exit openings 144 may be varied, as required in accordance with the arrangement of the unitary purification tanks such as in one, two or three dimensions. Alternatively, for example when configuring a 4-stage system, it is possible to configure the system such that a single unitary purification tank is used in stage I, three unitary purification tanks are used in stage II, two unitary purification tanks are used in stage III, and four unitary purification tanks are used in stage IV.

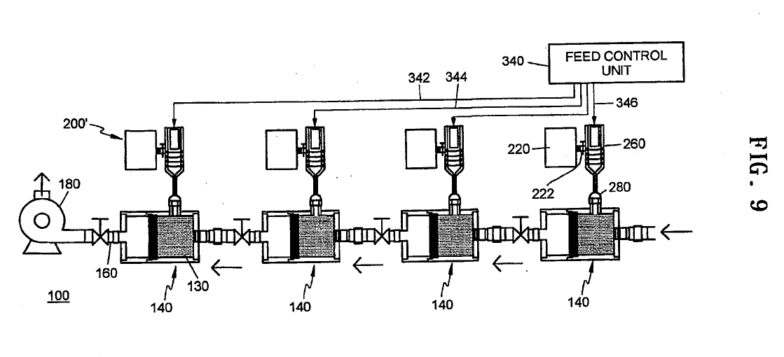

In addition, the unitary purification tank 140 may further comprise an injection opening 142 formed as a closable opening for injecting the purifier, such as the microorganisms and the culturing solution, through which the purifier is injected in the form of powder or liquid. With a manually operable injector 200 in the form shown in FIG. 5, a mixture of microorganisms with culturing solution in liquid state is sprayed into each unitary purification tank. In addition, onto the injection opening 142, an automatic feed apparatus to be described later with reference to FIG. 9 can be connected.

As shown in FIGS. 4 and 5, the multi-stage malodorous or noxious gas purification system in accordance with a preferred embodiment of the invention has a characteristic in that each unitary purification tank 140 is isolatedly connected one another via a connecting member or connecting tube 160. The connecting tube 160 is communicably installed at the inlet opening 143 or the exit opening 144 of each unitary purification tank 140, such that the two or more unitary purification tanks are isolatedly connected. Since it is desirable for the connecting tube 160 to allow gas-permeation only, an opening/closing switch 162 can be installed on the connecting tube 160 for better isolation, or it is possible to fill the connecting tube 160 with other filter material. The connecting tube 160 can be connected to an inlet portion 120 through which gas is introduced, with fastener members such as a joint nut 122, or can be used to connect different unitary purification tanks 140.

Another preferred embodiment of the invention may further comprise an air blower 180 for forcibly controlling the flow of the gas passing through the arrangement of the unitary purification tanks 140. The air blower 180 can be mounted at a rear end of the malodorous or noxious gas purification system as shown in FIG. 4, or can be mounted adjacent to each unitary purification tank 140 as shown in FIG. 5.

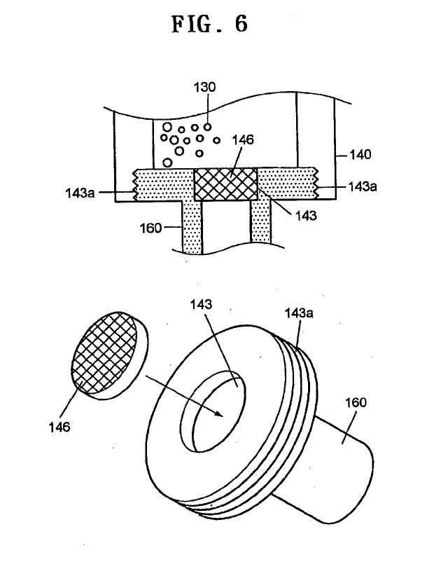

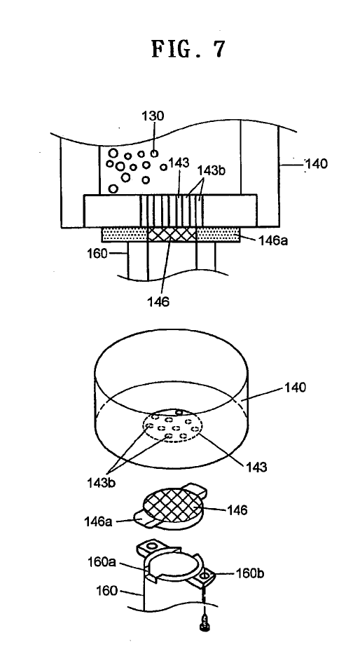

FIGS. 6 and 7 show a structure of a filter assembly of the unitary purification tanks in accordance with the invention, and the structure of the filter assembly facilitates replacement of the filters. With reference to FIG. 6, an inlet opening 143 is formed at a portion where one side of the unitary purification tank 140 containing the purifiers 130 connects with the connecting tube 160, and the part where the inlet opening 143 is formed may be integral with the connecting tube 160. The outer surface of the part where the inlet opening 143 is formed may be provided with threads 143a so as to be screwed into the unitary purification tank 140. In this structure, unscrewing the connecting tube 160 separates the inlet opening 143 from the unitary purification tank 140. Accordingly, the replacement of the inlet filter 146 inserted in the inlet opening 143 can be simplified.

In addition, with reference to FIG. 7, the inlet opening 143 formed onto the unitary purification tank 140 comprises a number of vents 143b. A filter frame 146a is attached to the inlet filter 146, and the connecting tube 160 comprises engageable grooves 160a for engaging with the filter frame 146a, and screwing portions 160b for screwing with the unitary purification tank 140. In this case, the connecting tube 160 and the inlet filter 146 are attached to the unitary purification tank 140 by and along with screws, and thus unfastening the screws on the connecting tube 160 makes replacement of the filter 146 easy.

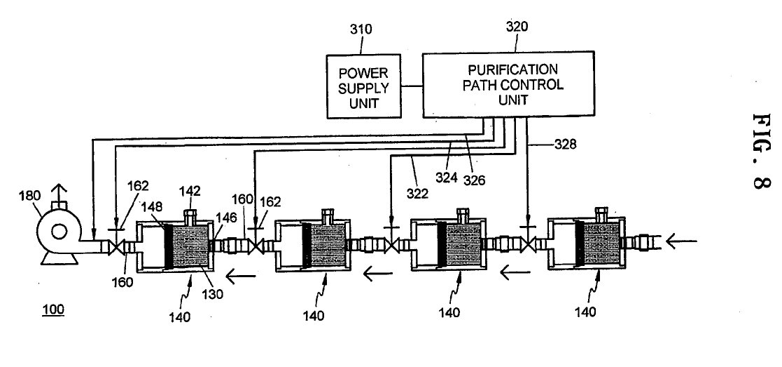

FIG. 8 shows a multi-stage malodorous or noxious gas purification system with a purification path control unit 320 in accordance with the invention. The purification path control unit 320 can control air flow by forcibly controlling the circulation rate of the air with the air blower 180, and can control the amount of air which remains in each unitary purification tank 140 and the time during which the air remains in the unitary purification tank 140 by respectively controlling opening/closing switches 162 installed at the connecting tubes 160. In this case, it is possible to optimize the conditions under which each unitary purification tank 140 can remove a different kind of causative agents of malodorous or noxious gas.

Such control is accomplished by controlling power supply from the power supply unit 310 through respective control lines 322, 324, 326 and 328 and controlling the operation of a motor of the air blower 180 and respective opening/closing switches 162, with the purification path control unit 320. The speed of the motor and the opening/closing time of the switches are determined empirically by taking the capability of the microorganisms being used and/or the concentration of the malodorous or noxious gas to be treated, etc. into account.

FIG. 9 shows a multi-stage malodorous or noxious gas purification system 100 with automatic feed apparatus 200′ for feeding microorganisms and culturing solution in accordance with a preferred embodiment of the invention. The automatic feed apparatus 200′ are connected to respective unitary purification tanks 140 and used for automatically feeding microorganisms and culturing solution containing organic substances as a nutritional source for the microorganisms. For the automatic feed apparatus, another prior art feed apparatus can be employed.

The automatic feed apparatus 200′ as schematically shown in FIG. 9 comprises a storage 220 for the microorganisms and culturing solution, and an injector 260 and nozzles 280 for discharging the microorganisms and culturing solution from the storage 220 into the unitary purification tank 140. With the operation of an opening/closing switch 222, a predetermined amount of the microorganisms and culturing solution can be fed from the storage 220 to the injector 260. The microorganisms and culturing solution fed into the injector 260 can be injected into the unitary purification tank 140 via the nozzles 280. Though such feed operation can be achieved manually, it is preferable to use a feed control unit 340 including control lines 342, 344 and 346 for such operation.

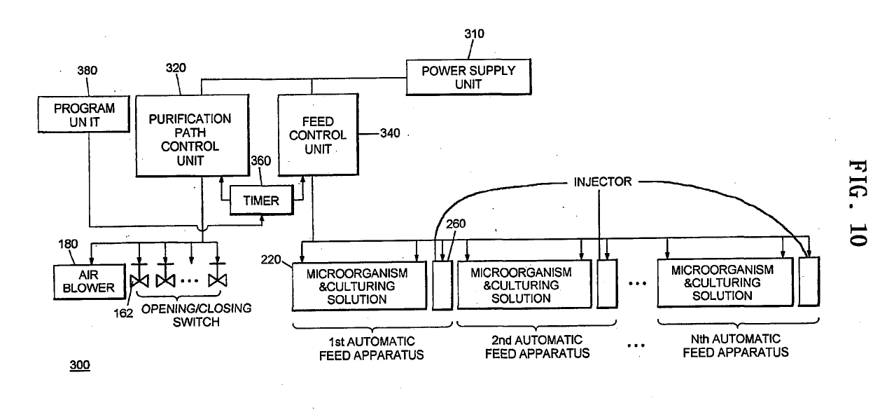

In the block diagram of FIG. 10 illustrating the control relationship of the multi-stage malodorous or noxious gas purification system in accordance with a preferred embodiment of the invention, it is shown that the purification path control unit 320 and the feed control unit 340 control the power supply to the motor and each opening/closing switch from the power supply unit 310, through time adjustment by a timer 360. The timer 360 in turn is configured to be automatically controlled by a program unit 380. Usually, the life expectancy of microorganisms is about four to five days, therefore, preferably the microorganisms and culturing solution are fed automatically in accordance with such life expectancy.

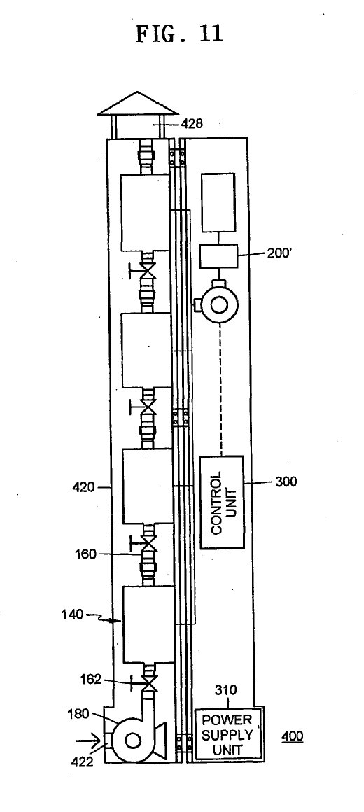

FIG. 11 shows a multi-stage malodorous or noxious gas purification system 400. configured in chimney type in accordance with an embodiment of the invention. The purification system 400 is a relatively small one among the examples which can be configured as a multi-stage malodorous or noxious gas purification system in accordance with the invention, and the purification system 400 comprises a chimney-type housing 420 to be adapted for outdoor mobile toilets or small waste treatment systems, etc. In the housing 420, malodorous gas is sucked by the air blower 180 at an inlet 422, the malodor is removed from the malodorous gas while passing through the arrangement of the unitary purification tanks 140, and discharged at an exit 428 as purified gas. Within the housing 420, an automatic feed apparatus 200′, a control unit 300, a power supply unit 310, etc. can be installed.

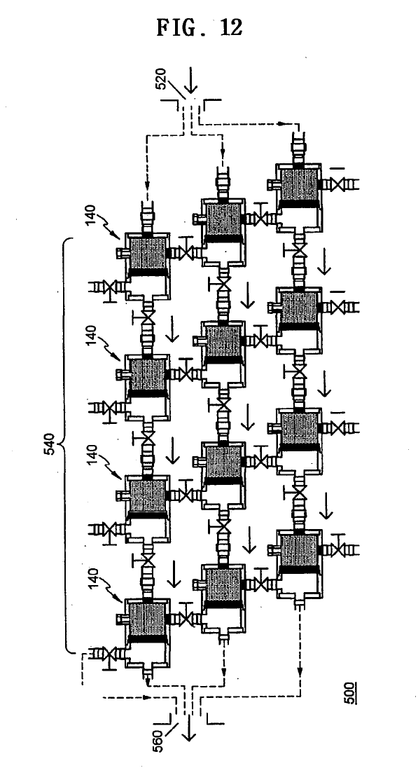

Though those embodiments of the unitary purification tanks 140 arranged serially or in one line are mainly described in the foregoing, the embodiments of the invention are not limited to such arrangement but it is also possible to arrange the unitary purification tanks 140 in two or three dimensions. FIG. 12 shows an exemplary configuration of a malodorous or noxious gas purification system 500 in two dimensional arrangement. The waste gas introduced through an inlet 520 is subjected to sequentially passing through a multitude of unitary purification tanks 140 in a purification section 540, and can be discharged at an exit 560 as purified gas with causative agents of malodorous gas removed. Accordingly, it is possible to configure a malodorous or noxious gas purification system that includes large facilities or that can treat a large amount of waste gas in accordance with such arrangement.

In accordance with a multi-stage malodorous or noxious gas purification system in accordance with the invention as described in the above, the present purification system can improve purification capacity over the prior art malodorous or noxious gas purification systems using purifiers or microorganisms. That is, the invention can provide a malodorous or noxious gas purification system with significantly increased purification capacity, since the causative agents of various malodorous or noxious gases can be removed sequentially and continuously from the gas including such causative agents. Such improvement of purification capacity is easily achieved by the malodorous or noxious gas purification system in accordance with the invention, in which each species of microorganisms that can decompose only certain causative agents of malodorous or noxious gas is isolatedly kept and waste gas is subjected to passing through habitat spaces accommodating such microorganisms.

The malodorous or noxious gas purification system in accordance with the invention as described in the above is highly flexible in its application due to the simple assembly of the system and the adjustability of scale as required. Accordingly, it is also possible to configure a small system such as an apparatus for removing the malodor from domestic electrical appliances such as refrigerators used at home. In addition, the present invention is also applicable to independent convenient facilities such as mobile toilets, and the invention can be employed even in environmental waste treatment facilities or for processing waste gas from industrial facilities, etc. if configured in large scale.

The present invention has been described in detail with reference to some preferred embodiments. However, it should be understood that the detailed description and specific examples are given by way of illustration only. Therefore, those skilled in the art will appreciate that various changes and modifications within the spirit and scope of the invention will become apparent from the detailed description.

1. A multi-stage malodorous or noxious gas purification system comprising:
purifiers for extracting unnecessary ingredients from gas including such unnecessary ingredients;
two or more unitary purification tanks each having a filling space for filling the purifiers therein and an inlet opening and an exit opening for the gas to pass through the filling space; and
at least one connecting member being gas-permeable and installed at the inlet opening or the exit opening of each unitary purification tank, for isolatedly connecting the two or more unitary purification tanks.

2. The multi-stage malodorous or noxious gas purification system as claimed in claim 1, wherein two or more different kinds of the purifiers are used.

3. The multi-stage malodorous or noxious gas purification system as claimed in claim 2, wherein at least one kind of the purifiers is a porous substance for adsorbing predetermined ingredients of the unnecessary ingredients.

4. The multi-stage malodorous or noxious gas purification system as claimed in claim 2, wherein at least one kind of the purifiers includes microorganisms for decomposing predetermined ingredients of the unnecessary ingredients.

5. The multi-stage malodorous or noxious gas purification system as claimed in claim 4, wherein the purifier including microorganisms comprises:
habitat members provided as environment for the microorganisms to live therein and also as the porous substance for adsorbing predetermined ingredients of the unnecessary ingredients;
microorganisms living in the habitat members;
and organic substances as a nutritional source for the microorganisms.

6. The multi-stage malodorous or noxious gas purification system as claimed in claim 5, wherein the nutritional source for the microorganisms is fed in liquid that includes the organic substances dissolved in a solvent.

7. The multi-stage malodorous or noxious gas purification system as claimed in claim 5, wherein the habitat members are porous carriers consisting mainly of carbon.

8. The multi-stage malodorous or noxious gas purification system as claimed in claim 5 or 7, wherein the habitat members are micro-porous carriers having fine pores formed therein, the size of the fine pores being about a few microns in diameter.

9. The multi-stage malodorous or noxious gas purification system as claimed in claim 1, wherein each unitary purification tank comprises a gas-permeable intermediate filter for dividing the filling space into at least two partitioned spaces, and one of the two partitioned spaces is filled with the purifiers.

10. The multi-stage malodorous or noxious gas purification system as claimed in claim 9, wherein each unitary purification tank comprises a gas-permeable inlet filter at the inlet opening, and a space between the intermediate filter and the inlet filter is filled with the purifiers.

11. The multi-stage malodorous or noxious gas purification system as claimed in claim 9, wherein each unitary purification tank has one or more inlet openings at the space filled with the purifiers and one or more exit openings at a space with no purifier filled therein.

12. The multi-stage malodorous or noxious gas purification system as claimed in claim 1, wherein each unitary purification tank is further provided with a closable injection opening for injecting the purifier.

13. The multi-stage malodorous or noxious gas purification system as claimed in claim 1, further comprising an apparatus for automatically feeding microorganisms and culturing solution including organic substances as a nutritional source for the microorganisms, each apparatus connected to respective unitary purification tanks.

14. The multi-stage malodorous or noxious gas purification system as claimed in claim 1, wherein each connecting member comprises a connecting tube one end of which is communicably connected to an inlet opening of one unitary purification tank and the other end of which is communicably connected to an exit opening of. another unitary purification tank, and a switch for opening and closing the connecting tube.

15. The multi-stage malodorous or noxious gas purification system as claimed in claim 1, further comprising an air blower for forcibly controlling the flow of the gas passing through the unitary purification tanks.

16. The multi-stage malodorous or noxious gas purification system as claimed in claim 1, wherein the unitary purification tanks are arranged serially in one dimension, a chimney-type housing is further provided for the protection of the serially arranged unitary purification tanks, and an apparatus for automatically feeding the microorganisms and culturing solution into each unitary purification tank is installed within the chimney-type housing.

17. The multi-stage malodorous or noxious gas purification system as claimed in claim 1, wherein the unitary purification tanks are arranged in two or three dimensions.

18. A multi-stage malodorous or noxious gas purification system for removing unnecessary ingredients from gas including such unnecessary ingredients, the purification system comprising:
two or more different kinds of purifiers for extracting the unnecessary ingredients from the gas;
a plurality of unitary purification tanks each having a filling space for filling the purifiers therein and an inlet opening and an exit opening for the gas to pass through the filling space; and
at least one connecting member being gas-permeable and installed at the inlet opening or the exit opening of each unitary purification tank, for isolatedly connecting the plurality of unitary purification tanks,
wherein at least one kind of the purifiers comprises microorganisms for decomposing predetermined ingredients of the unnecessary ingredients, habitat members of a micro-porous substance consisting mainly of carbon and having fine pores formed therein about a few microns in size for providing environment for the microorganisms to live therein, and organic substances in liquid state as a nutritional source for the microorganisms,
wherein each unitary purification tank is provided with a gas-permeable intermediate filter for dividing the filling space into at least two partitioned spaces, and one of the two partitioned spaces is filled with the purifiers,
wherein the purification system further comprises an apparatus connected to respective unitary purification tanks for automatically feeding the microorganisms and culturing solution including organic substances as a nutritional source for the microorganisms, and an air blower for forcibly controlling the flow of the gas passing through the unitary purification tanks.
